(19) United States
(12) Patent Application Publication
(10) Pub. No.: US 2026/0133119 A1
(43) Pub. Date: May 14, 2026

(54) IMAGING SYSTEM FOR AQUATIC ORGANISMS

(71) Applicant: SINTEF TTO AS, Trondheim (NO)

(72) Inventors: Bjarne KVÆSTAD, Trondheim (NO); Trond NORDTUG, Jakobsli (NO)

(21) Appl. No.: 19/118,066

(22) PCT Filed: Oct. 4, 2023
(86) PCT No.: PCT/NO2023/060063

§ 371 Date: Apr. 3, 2025

(30) Foreign Application Priority Data
Oct. 5, 2022 (NO) 20221060

(51) Int. Cl. G01N 15/1434; A01K 61/95; G01N 15/01; G01N 15/14

(52) U.S. Cl. CPC G01N 15/1436; A01K 61/95; G01N 15/01; G01N 2015/1493

(57) ABSTRACT

An imaging system for imaging aquatic organisms. The imaging system includes an imaging device and at least two mirrors arranged to provide imaging of the aquatic organisms from at least two angles. The imaging system may be provided with two illumination sources. The imaging system may image small aquatic organisms while they pass through a flow cell.

### INTRODUCTION

The present invention concerns monitoring of small aquatic organisms. In particular, the invention relates to an imaging system for imaging of small aquatic organisms. The imaging system may be a part of a flow-through imaging system. The imaging system and the flow-through imaging system may be used for repeated monitoring of e.g. growth and health status of small aquatic organisms.

### BACKGROUND

Monitoring of small aquatic organisms are important for a number of reasons. There is an increased awareness of the importance of monitoring the ocean ecosystem throughout the entire food chain. Preserving the ocean and the ocean ecosystems is important for the preservation of life on earth and the diversity in nature as well as providing food for a growing human population. Small aquatic organisms represent an important part of the food chain in the ocean and its of interest to monitor e.g. morphometrics, growth and density of the small aquatic organisms in the ocean. In e.g. fish farming monitoring is important both of fish larvae and feed for the fish larvae, as e.g. zooplankton. Examples of monitoring may e.g. be related to morphometrics of small aquatic organisms, how their size changes over time, how the aquatic organisms grow and develops, the density of the aquatic organism in a fish tank or a fish farming sea pen and classification of aquatic organisms present in the ocean or tank. It is well established that unfavorable and toxic environments during the embryonic stage of fish increases the frequency of developmental malformations in larvae after hatching and this may be used in ocean monitoring as an indicator water quality.

As a further example, in the breeding of marine fish species, the spawning phase is the most demanding, and this phase is still the largest bottleneck from a production perspective. This is because newly hatched fish larvae are very small, and the density of spawn in each tank is high. Marine fish larvae are poorly developed after hatching and, unlike salmon, need good quality living feed organisms to grow and develop normally. The period of initial feeding, from the time the larvae begin to eat until they are large enough to be able to digest formulated feed, is often characterized by high mortality and incorrect development. How well the larvae perform in a farming environment depends on many complex factors such as feed quality, water quality and quality of the roe.

Monitoring fish growth is important. Good early nutrition is crucial for how the fish grow and develop later in life. The fish larvae are monitored at an early stage in order to achieve predictability and profitability later in production. The growth, development and appetite (stomach filling) of fish larvae is most often monitored by larvae being taken out of the tub and examined under a magnifying glass/microscope where they are measured and scored. Despite that a breeder can spend 2-3 work months a year on this type of task, this still provides far too little information for the breeders to feel it is worth the time invested. The low number of larvae can give non-representative targets for the population, and there are currently no good systems for data logging. This means that groups in different vessels in the same installation, or groups from year to year, cannot be compared. This also means that underperforming groups are detected too late to be able to take measures.

There is a need for a simple and cost-efficient monitoring system for small aquatic organisms, that may be easily adapted to different species of aquatic organisms and environmental conditions.

### SUMMARY OF THE INVENTION

The invention provides a solution to or at least alleviates some of the problems mentioned above.

The invention provides an imaging system enabling imaging of small aquatic organisms from at least two angles. The imaging system comprises an imaging device and at least two mirrors. The imaging device and the at least two mirrors are arranged to provide imaging of the at least one aquatic organism from at least two angles.

The invention also provides a flow-through imaging system for imaging at least one aquatic organism. The at least one aquatic organism may be imaged while the at least one fish larvae pass through a flow cell. The flow-through imaging system may comprise a flow cell, and an imaging system according to above. The invention also provides uses of the imaging system and flow-through imaging system.

The invention provides an imaging system for imaging at least one aquatic organism. The imaging system comprising an imaging device, and at least two mirrors, wherein the mirrors are arranged to provide imaging of the at least one aquatic organism from at least two angles.

The imaging system may further comprise at least two illumination sources. The at least two illumination sources may be arranged on opposite sides of the imaging device. The imaging device may be arranged on a first side of the at least one aquatic organism and the at least two mirrors may be arranged on a second side of the at least one aquatic organism, wherein the first side is opposite the second side. The at least two mirrors may be arranged to obtain at least two reflections from the at least one aquatic organism. The at least two mirrors and the imaging device may be arranged to obtain a combined image of the at least one aquatic organism from three different angles, where the first and second angles are provided by reflections from the at least one aquatic organism with the at least two mirrors and into the imaging device and the third angle is provided by a direct image of the at least one aquatic organism into the imaging device.

The at least two mirrors may each be arranged at an angle relative to a plane perpendicular to the axis of a field of view of the imaging device, wherein the angel is approximately from 15° to 45°.

The at least two mirrors may be arranged approximately 22.5° relative to a plane perpendicular to the axis of field of view of the imaging device to obtain two reflections from the aquatic organism of approximately 90° relative to each other. Each of the at least two mirrors may alternatively be arranged at an angle of approximately 30° relative to a plane perpendicular to the axis of the field of view of the imaging device. The at least two mirrors may be arranged to obtain a combined image of the at least one aquatic organism provided by views from three different angles, where the three different views are at an angle of 120° relative to each other, wherein two of the angles are reflections from the aquatic organism via the at least two mirrors and into the imaging device and a third angle is a direct image of the aquatic organism into the imaging device. The at least two mirrors may be symmetrically arranged, or nearly symmetrically arranged, relative to the axis of the field of view of the imaging device. The at least two mirrors may be arranged to enable a same focus area for the imaging device for the viewing angles. The imaging device may include a telecentric lens. The images of the at least two angles/sides of the at least one aquatic organism may be obtained simultaneously.

The at least one aquatic organism may be arranged to flow through a flow cell while being imaged by the imaging device. The flow cell and the at least two mirrors may be arranged inside a container filled with transparent material. The container may be filled with a degassed liquid or a clear resin. The imaging device and the at least two illumination sources may be arranged on the inside or the outside of the container. The flow cell and the container may be transparent. The container may be made of polycarbonate or quartz. The flow cell may be made of polycarbonate, quartz or plastic. The flow cell may further comprise at least one optical diffuser.

The invention also provides a flow-through imaging system for imaging at least one aquatic organism while the at least one aquatic organism passes through a flow cell. The system comprising a flow cell, and an imaging system. The imaging system may be according to above. The flow cell and the at least two mirrors may be arranged inside a container filled with a liquid or clear resin to remove optical distortions caused by irregularities in the flow cell. The flow-through imaging system may further comprise a valve for adjustment of a flow through the flow cell.

The invention also provides uses of the imaging system for monitoring size or growth of small aquatic organisms. The imaging system or the flow-through imaging system may also be used for constructing 3D images of small aquatic organisms or for morphometric measurements of small aquatic organisms. The flow-through imaging system may also be used for monitoring growth of fish larvae, or for monitoring a health status of fish larvae, or for biometric measurements of fish larvae or for monitoring of fish larvae in production tanks. The small aquatic organism may be at least one of a fish larva, zebrafish larva, algae, crustacean, zooplankton or an egg from an aquatic organism.

The imaging system provides a flexible solution for imaging aquatic organisms. The imaging system is easily scalable for imaging aquatic organisms having different sizes and shapes and mutual arrangement between the components of the system. The imaging device and mirrors and possible light sources may be separately arranged independent of each other and may be arranged closer or more far apart. The mirrors may have different areas and shapes as long as the ability of the imaging system for providing imaging of the aquatic organism from at least two angels remains. An imaging system arranged external to a possible flow cell enabling flexibility concerning e.g. size, shape and mutual arrangement of the imaging device and mirrors and possible light sources as explained above. The imaging system with or without flow cell also allow for easy exchange or repair of the individual components of the system. The mirrors and the flow cell arranged inside a container may also allow for easy exchange of the container with flow cell and mirrors for adapting the imaging system for a different use e.g. for monitoring other aquatic organisms or for easy exchange of the mirrors and flow cell in case of damage or wear. The new container may be easily connected to the illumination sources and the imaging device which may be arranged on the inside or the outside of the container.

The imaging system is also easily adaptable for different uses e.g. in the ocean, in fish farming or in closed systems both in the sea and on land. The imaging system may e.g. be used for establishing the density of algae in the ocean, classification of algae in the ocean, for morphometric measurements of crustaceans in the open oceans, establishing growth of fish larvae, the quality of the fish larvae, measuring the size of the yolk sac versus length, identifying any deformations of the larvae. Other uses may be in monitoring production of live feed e.g. of zooplankton, fish larvae, algae and egg where morphometric measurements may establish growth, density, quality etc.

As an example only, the imaging system may enable automatic imaging and analysis of fish larvae. The fish larvae may e.g. be taken out of fish tanks and made to flow through the imaging system through a flow cell. The fish larvae images may be analyzed using machine learning. The imaging system with flow cell enables automatic biometric measurements of the fish larvae. The images may be analyzed in real time. The images also enable later 3D reconstruction of the larvae at a later time. The flow through imaging system provides an effective and more accurate system for imaging of large amounts of fish larvae without affecting the health and viability of the fish larvae. Thus, the system may be used for repeated analyses of the same cohort of larvae over time. The example for the fish larvae above may also apply to other small aquatic organisms. The imaging system is for imaging small aquatic organisms as e.g. exemplified in the application, but may also apply to small fish and other small living aquatic organisms.

### BRIEF DESCRIPTION OF DRAWINGS

Example embodiments are described with reference to the following drawings, where:

FIG. 1 is an illustration of an exemplary imaging system for imaging of at least one aquatic organism from two angles;

FIG. 2 is an illustration of another exemplary imaging system for imaging at least one aquatic organism from three angles;

FIG. 3 is an illustration of an exemplary imaging system for imaging of at least one aquatic organism from two angles;

FIG. 4 is an illustration of an exemplary imaging system arranged in relation to a tank for aquatic organisms for monitoring the aquatic organisms in the tank;

FIG. 5a is a schematic view of an exemplary imaging system for imaging of fish larva,

FIG. 5b is a schematic view of the exemplary imaging system for imaging of fish larva from FIG. 5a showing the imaging device, mirrors, flow cell and illumination sources in a side and front view,

FIG. 6 is an example showing three images of the same zebrafish larva taken with an imaging system as illustrated in FIGS. 5a and 5b. The images of the zebrafish larvae on the left and right are images taken via the mirrors on the right and left side and are rotated 90° relative to each other. The image in the middle is taken directly by the larva inside the flow cell.

### DETAILED DESCRIPTION

Example embodiments are described with reference to the drawings. The same reference numerals are used for the same or similar features in all the drawings and throughout the description. The examples are illustrations only and are not limiting for the invention.

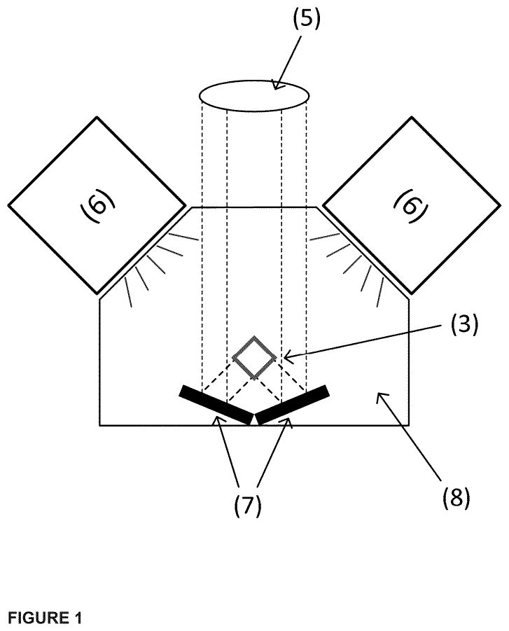

An imaging system 1 for imaging at least one aquatic organism is illustrated in FIG. 1. As illustrated in FIG. 1 the imaging system includes an imaging device 5 and two mirrors 7 The two mirrors 7 are arranged to provide imaging of an aquatic organism from two angles (two-angle system). The imaging of the aquatic organism from two angles is performed with one imaging device. The imaging of the aquatic organism from two angles may be performed simultaneously. Imaging both sides of the aquatic organism simultaneously increase the chances of getting a good picture of the aquatic organism that may be used for automatic biometric measurements and offer the possibility of 3D reconstruction of the aquatic organism at a later time.

The imaging system has a flow cell 3 for flow through of the aquatic organism to be imaged. In FIG. 1 the flow cell 3 and mirrors 7 are arranged inside a clear container 8. The container may be filled with transparent material. The container may be filled with a degassed liquid or a clear resin. The container may also be cut out of a solid piece of polycarbonate, quartz or other transparent solid material. The container may be filled with e.g. degassed freshwater or clear mineral oil. The container may also be moulded with clear resin. The mirrors in FIG. 1 are arranged to provide reflections from the aquatic organism in the flow cell 3 where the reflected light rays are parallel with each other when entering the imaging device 5 as illustrated in FIG. 1. The reflected light rays are also parallel with the optical axis of the imaging device. The mirrors in FIG. 1 of the imaging system may be arranged to obtain two reflections from the aquatic organism of approximately 90 degrees relative to each other. In FIG. 1, each of the mirrors are arranged at an angle of approximately 22.5° in relative to a plane perpendicular to the axis of field of view of the imaging device. The two mirrors are arranged on each side of the center axis of the field of view of the imaging device. The first mirror is arranged on the left side of the center axis as illustrated in FIG. 1. The second mirror are arranged on the right side of the center axis as illustrated in FIG. 1. The light opening (lens) of the imaging device is arranged in parallel with the inside bottom of the container as shown in FIG. 1. The angle of the mirrors of approximately 22.5° is therefore also defined in relation to the inside bottom, of the container as shown in FIG. 1.

The imaging systems in FIG. 1 may also be provided with optional light/illumination sources for illumination of the flow cell. FIG. 1 has two illumination sources. One illumination source 6 is arranged on opposite sides of the imaging device 5. The light sources are arranged to illuminate the flow cell from partly above and from the side. The light sources are attached to the outside of the container 8. The container is provided with cut off corners at the upper edges of the container for attachment of the light sources to the outside of the container. In the illustrated embodiment in FIG. 1 the light sources are symmetrically arranged, or almost symmetrically arranged, in view of the imaging device and downwardly inclined to illuminate the flow cell from partly above and from the side. Use of the light sources may increase the quality of the images by increasing the contrast in the image and avoiding shadows in the image. This improves the contrast between the aquatic organism and the surrounding water and may also increase the contrast between the different parts of the aquatic organisms both on the outside and of internal structures of transparent or partly transparent aquatic organisms. The illumination sources may be separately exchangeable.

In FIG. 1 the at least two mirrors 7 are illustrated to be attached to the inside of the container 8. Alternatively, the mirrors 7 may also be attached to a first frame attached to the inside of the container. The angle of the mirrors may be fixed or arranged to be variable. The flow cell 3 may also be attached to a frame attached to the inside of the container. The frame for the mirrors may be part of the same frame structure to which the flow cell is attached. The flow cell may however also be integrated with the mirrors and the combined integrated part be attached to the inside of the container either directly or by a frame structure. The two mirrors may also not be separately attached to the inside of the container, but may be attached to each other forming a mirror unit with two reflecting mirror surfaces. The mirror unit may also be attached to the flow cell forming a single unit. The mirror unit and the flow cell may separately exchangeable, and the combined mirror and flow cell unit may also be exchangeable. The container with mirrors and flow cell unit may also be exchangeable as one unit. The imaging device and possible illumination sources external to the container may easily be disconnected from and connected to any container. The flow cell may also be easily disconnected from a possible flow through tube(s) for inflow and outflow of the small aquatic organisms to and from the flow cell inside the container. The aquatic organism may be transported passed the imaging system through the flow cell one by one or many at the same time. The imaging system may image one or more aquatic organisms at the same time.

The upper side and the lower side are used as an explanation only in relation to FIG. 1 and upper and lower is seen in relation to the flow cell inside the container. The mirrors are arranged under the flow cell and the imaging device above the upper side of the flow cell. The container with the imaging system may however be arranged in any orientation in the ocean, in a fish farm cage etc.

In FIG. 1 the imaging device is arranged above the aquatic organism. As explained above, the imaging system may be arranged in any orientation. The imaging device 5 is arranged on a first side of the aquatic organism. The at least two mirrors are arranged on a second side of the at least one aquatic organism, wherein the first side is opposite the second side. The at least two mirrors are arranged to obtain at least two reflections from the at least one aquatic organism providing imaging of the at least one aquatic organism from at least two sides. The flow cell in FIG. 1 is shown having a square cross section, but other shapes may also be possible.

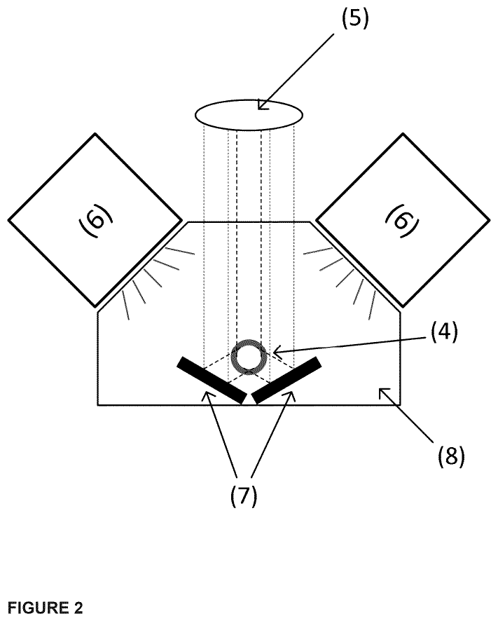

FIG. 2 illustrates another example of an imaging system for imaging of an aquatic organism. The at least two mirrors and the imaging device may be mutually arranged to obtain an image of the at least one aquatic organism from three different angles (three-angle system). The first and second angles are provided by reflections from the at least two mirrors and into the imaging device and the third angle is provided by an image from the at least one aquatic organism and into the imaging device. The imaging system has the same main components as in FIG. 1 as explained in detail above. The flow cell in FIG. 2 has a circular cross section. The arrangement of the flow cell, mirrors, lens and lights enables imaging of the fish larvae from three angles using only one imaging device.

The at least two mirrors may be arranged to obtain a combined image provided by reflections from three different directions/angles. The three different directions may be at an angle of about 120° relative to each other (three-angle system). Two of the directions are reflections from the aquatic organism via the at least two mirrors and into the imaging device. Light is reflected from the aquatic organism inside the flow cell 3 and onto the mirrors 7 and then reflected from the mirrors and into the imaging device 5. A third direction is a direct image of the aquatic organism into the imaging device. The mirrors are arranged at a different angle with respect to the bottom of the tank and thereby also with respect to the plane provided by the imaging device and lens opening. The flow cell has a circular cross-section in FIG. 2 rather than a square cross-section as in FIG. 1. The angle of the mirrors is set to 30° relative to the inside bottom to produce view angles from three directions separated by 120° of the aquatic organism and into the imaging device 5. The reflected light rays may arrive at the imaging device in parallel as illustrated in FIG. 2 to provide a combined single image from three view angles.

The use of at least two mirrors enables the imaging device to have the same focus area for the objective for both viewing angles. This enables use of an imaging device with a telecentric lens. The telecentric lens may be used for all viewing angles. The arrangement of the mirrors also provides the light travel path from the aquatic organism and into the imaging device from each of the mirrors to be the same or substantially the same. The aquatic organism would then appear as having the same size for all viewing angles. This enables more accurate size measurements and/or 3D reconstruction of the aquatic organism. It is desirable that the images obtained from the at least two sides of the aquatic organism are symmetrically or substantially symmetrical.

In the examples above, the mirrors are shown with two different angels in view of a plane perpendicular to the axis of the field of view of the imaging device. The axis of the field of view of the imaging device correspond to the optical axis of the imaging device. Other angels are also possible. Each of the mirrors may also be arranged at an angle from approximately 15° to 45° relative to a plane perpendicular to the axis of field of view of the imaging device. An angle for the mirrors may more preferably be in the range from approximately 25° to 35°. The mirrors may be symmetrically arranged, or nearly symmetrically arranged relative to the plane perpendicular to the axis of the field of view of the imaging device.

The mirrors may be a surface coated with a reflective material which can reflect a clear, detailed image of an object.

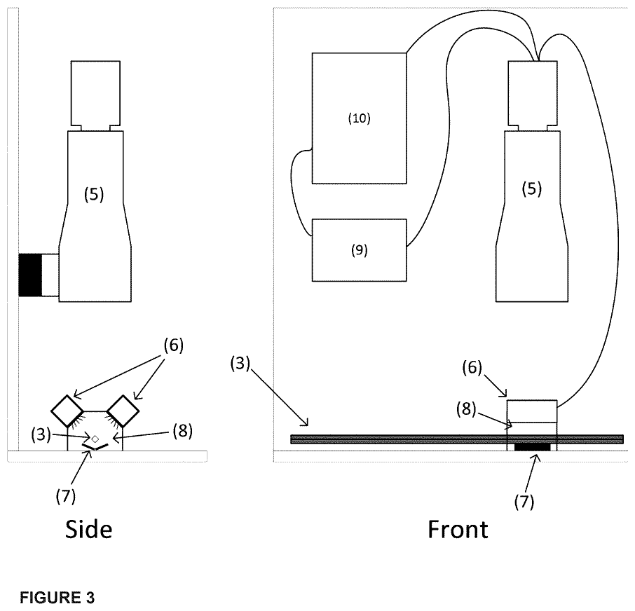

FIG. 3 illustrates an imaging system for imaging aquatic organisms. The imaging system comprises an imaging system arranged for imaging fish larvae inside a flow cell. The imaging device is arranged above the flow cell. The imaging system is provided with two light sources illuminating the flow cell. The two light sources are arranged on opposite sides of the imaging device. The two mirrors are arranged on the underside of the flow cell as illustrated in FIG. 1. Each of the mirrors is arranged at an angle in relation to the longitudinal direction of the flow cell or the plane provided by the lens opening of the imaging device 5. The mirrors are arranged to provide two reflections relative to each other so that the aquatic organism may be imaged from two directions/sides. The imaging from the two angles may be performed at the same time. The at least two sides of the aquatic organism may be about 90° in relation to each other. The mirror, flow cell, imaging device and illumination devices from the example in FIG. 2 may be implemented in FIG. 3 instead of the example configuration of the mirrors, flow cell, imaging device and illumination devices from FIG. 1. In an example where the imaging system from FIG. 2 is implemented in FIG. 3, the three different directions may be at an angle of about 120° relative to each other (three-angle system). Further embodiments imaging the aquatic organism from more than three angles may also be envisaged. The arrangement of the flow cell, mirrors, lens and lights enables imaging of the fish larvae from at least two angles using only one camera. The imaging of the aquatic organism from at least two directions may be performed simultaneously or almost simultaneously.

FIG. 3 shows the imaging system and also a computer 9 and power supply 10. The computer may control the imaging system with imaging device and flow cell. The computer 9 may also optionally control through-flow of the aquatic organisms through the flow cell. Through-flow of the aquatic organisms through the flow cell may however e.g. be controlled by a separate control device or may e.g. be implemented by a natural flow. The computer may also store imaging data and analyse the imaging data taken with the imaging device 5. Image analysis may also be performed in a remote location. The imaging system may be provided with a transmitter for transmitting imaging data to a remote location in almost real time. The imaging system may alternatively also be provided with an interface for later downloading of imaging data taken with the imaging device. The imaging system may also be provided with an interface for external communication with the imaging device. External communication with the imaging device may e.g. take place on site e.g. also in an underwater location.

The imaging system with imaging device, flow cell, mirrors, illumination devices, computer and power supply in FIG. 3 are arranged inside a container. The container may be transparent. The container may be filled with a transparent material. The container may e.g. be filled with a degassed liquid or a clear resin. The transparent material may remove optical distortions caused by irregularities in the flow cell. The imaging device 5 in FIG. 3 is attached to an inside wall of the container. The mirrors, with flow cell and illumination devices are attached to the inside bottom of the container. The imaging device may be a camera, e.g. an infrared camera or a camera operating in the UV wavelength or a camera operating in the visible wavelength range or a combination of UV and visible wavelengths. The camera may record images one at the time, but may also record video. The illumination sources may be in the visible parts of the wavelength range, but may also emit light in the UV wavelength range. Some aquatic organisms might emit fluorescent light when irradiated by the illumination devices and the fluorescent light emitted from the aquatic organisms detected by the imaging device. The at least two illumination sources 6 and the at least two mirrors 7 are arranged on the outside of the flow cell 3. The flow cell may be made of transparent material such as e.g. polycarbonate, quartz, plastic. The transparent material allows the aquatic organism to be imaged without them being removed from the water. The flow cell illustrated in FIG. 3 has a cross-sectional shape of a square. The flow cell in FIG. 3 is arranged with each of the two adjacent upper longitudinal sides facing the two light sources enabling at least some of the light from the light sources to pass directly through the flow cell without being refracted. The light sources are meant to illuminate the aquatic organism from all angles, where some of the light are shined directly on the aquatic organism and some lights are illuminating the aquatic organism via the mirrors 7, giving an image with both a clear contrast and a clear Silhouette boundary. The light then enters the imaging device 5. At least some other parts of the light reflected by the mirrors 7 may travel directly into the imaging device 5. The flow cell may be provided with an optical diffuser on the outside of at least some of the flow cell sides, preferably the flow cell sides facing the light source, for scattering the light entering the flow cell providing a softer light for imaging the fish larvae. The imaging device and the at least two illumination sources may also be arranged on the outside of a container. Also, the computer and power supply may also be arranged on the outside of the container.

In FIG. 3 each of the mirrors are arranged at an angle of approx. 22.5° in relation to a longitudinal direction of the flow cell/the inside bottom of the container/the imaging plane of the lens of the imaging device. The mirrors may also be arranged in other angles, e.g. at 30° in relation to a longitudinal direction of the flow cell. In FIG. 3 the at least two mirrors 7 and the imaging device 5 may be mutually arranged to obtain an image of the aquatic organism in the flow cell from at least two different directions. The first and second directions are provided by reflections from the first and second mirrors respectively. A third direction may be provided by a direct image of the aquatic organism and into the imaging device if a set-up of the mirrors and flow tube from FIG. 2 is used. The mirrors may then e.g. be arranged in approximately 120° relative to each other. Other shapes of the flow cell may also be possible e.g. a rectangular shape, a an oval shape or a circular shape.

The flow cell and the flow system are designed so as to cause no harm to the aquatic organism. Further light sources may also be provided to improve the images of the aquatic organisms. Further light sources may increase the contrast and reduce shadow effects in the images. The light sources are arranged to enable imaging of the aquatic organism from at least two sides. The camera, mirrors and light sources are arranged on the outside of the flow cell. This provides flexibility to the arrangement of the camera, mirrors and light sources both in view of the flow cell and with respect to their mutual arrangement. The mirrors may be arranged at different angles as explained above adapted to the actual flow cell used and the system requirements. The external arrangements outside the flow cell also provides increased flexibility for the exchange of the camera, mirrors and light sources for repair or exchange or for exchange of one or all of camera, mirrors and light sources to adapt to the particular flow cell. The size and/or shape of the flow cell, the camera used and/or the mirrors used, may be adapted to aquatic organisms with different dimensions. The imaging system may also be provided with instrumentation for storage and processing of data. Automatic image processing for biometric measurements may be provided. FIG. 3 may be implemented as an ex-situ imaging system providing an “all in one” system enabling both imaging and analyses of the images. The processing may be performed in real time or by post processing of data.

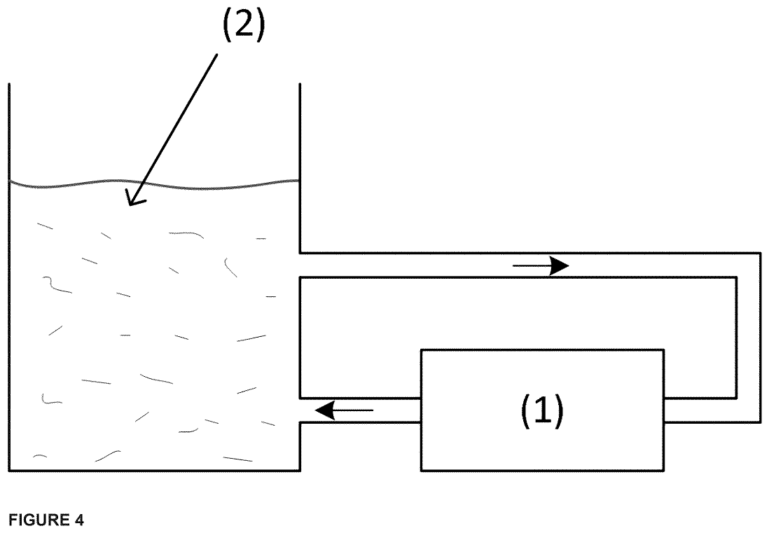

FIG. 4 illustrates an imaging system arranged in relation to a tank 2 with aquatic organisms, e.g. fish larvae, zebrafish larva, zooplankton. For imaging of the aquatic organisms these are made to flow out of the tank through e.g. a pipe or flow tube and to the imaging system 1. In the imaging system, the aquatic organisms are imaged while they are passing through the imaging system. The imaging system is adapted to the specific use and the aquatic organism in the tank. In addition to the components of the imaging system as illustrated in FIG. 1 or FIG. 2, the imaging system in FIG. 4 may e.g. also be provided with a valve for flow adjustment, and optionally a trigger system for the imaging system. The trigger system may detect the aquatic organisms before arrival at the imaging system and the imaging system activated at the right time so that imaging may take place with the aquatic organism centred in the image while the aquatic organism passes through the flow tube imaging system. The flow tube is provided with the imaging system with a flow cell for imaging each of the aquatic organism while the aquatic organism passes through the flow cell. The imaging system may image the aquatic organism from two or more angles. The imaging from two or more angels may be performed at the same time. More than one aquatic organism may be imaged at the same time. After passing the imaging system, the at least one aquatic organism is passed further on in the flow tube and into the tank as illustrated in FIG. 4. The imaging system is a flow-through imaging system. The flow tube is designed to provide transport of the aquatic organism passed the imaging system with flow cell. The aquatic organism may be transported passed the imaging system through the flow cell one by one or many at the same time.

The aquatic organism may alternatively end up in a second tank after passing through the flow through imaging system. A valve of the flow tube may be provided for controlling the flow velocity of the flow of aquatic organism through the flow tube and imaging system. The flow velocity should be adapted to enable the aquatic organism to be transported through the imaging system slowly enough to be imaged by the imaging system one by one. A number of aquatic organisms may also be imaged together.

FIG. 4 is an illustration only and other tank configurations may be possible. The flow tube may end up immersed in water. There may be embodiments where the aquatic organism fall down into a second tank. The first tank may be in an elevated position in view of the second tank to enable flow of an aquatic organism from the first tank to the second tank under the force of gravity.

Using the force of gravity for transporting the aquatic organism through the flow-through imaging system in FIG. 4 is also provided in the system in FIG. 4 as the aquatic organisms are taken out from the tank and into the flow through tube at a level above the return level of the flow through tube. Use of the force of gravity provides a good solution also taking good care of the welfare and health of the aquatic organism. Use of gravity provides a simple system where the aquatic organisms do not need to pass through other equipment e.g. pumps. The use of pumps may potentially provide an increased risk for injury to the aquatic organism and also increases the costs of the system. It is however possible to provide embodiments involving use of pumps to enable the flow of aquatic organisms to pass through the flow tube when flow under the force of gravity may not be possible.

### Example

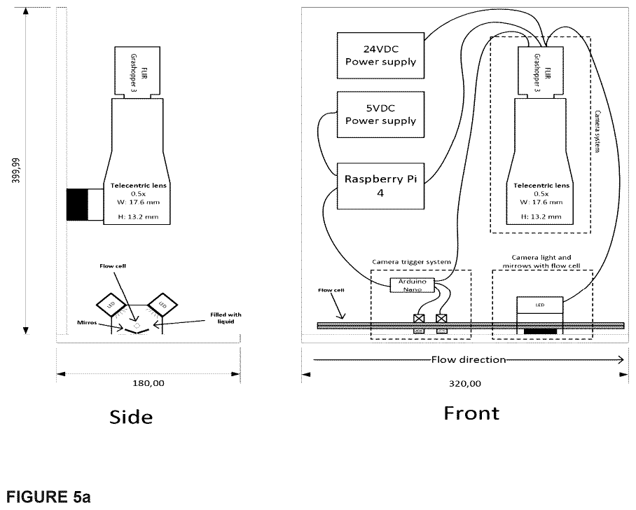

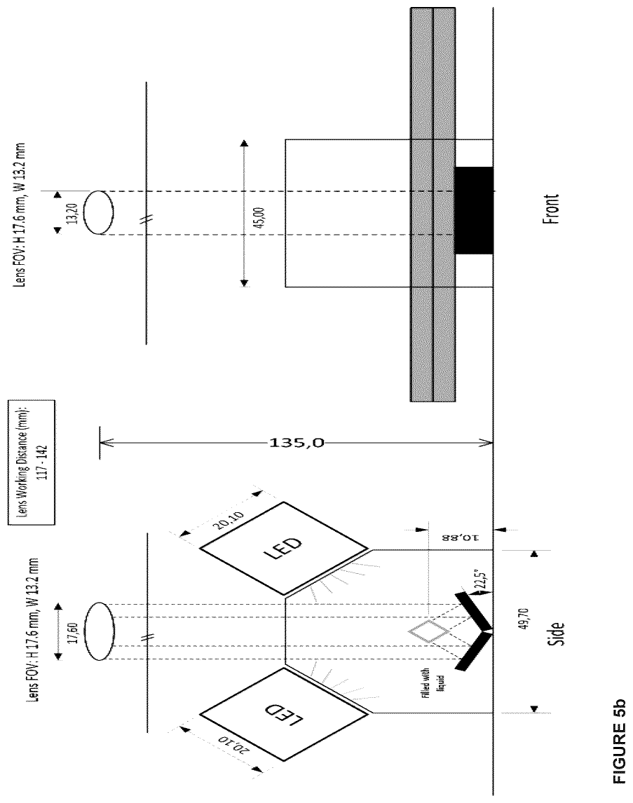

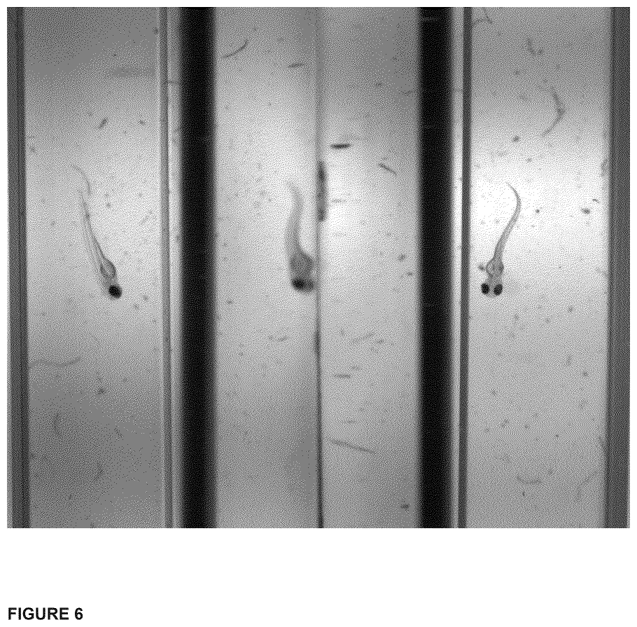

An example of an imaging system with a flow cell used in an experimental setup for obtaining images of a fish larvae as shown in FIG. 6 is illustrated in FIG. 5a. FIG. 5a may illustrate a schematic of an experimental setup of the imaging system with components with dimensions and location according to the various data sheets for the components. The trigger system illustrated is only optional. The imaging system in FIG. 5 a is optimized for 3-10 mm long fish larvae with a maximum diameter of 4 mm. It may be possible to adjust the imaging system to image larvae from 3-30 mm, but this may reduce the resolution for larvae from 3-10 mm.

In the example in FIG. 5a the flow cell has a square cross-section and has an inner width of 4 mm and is approx. 300 mm long. The flow cell and mirrors are provided inside a container filled with liquid. The mirrors used were 50 ×75 mm Enhanced Aluminum, 4-6λ Mirror from Edmund optics. Two illumination sources were provided on the outside of the container. Further details of the flow cell are shown in enlarged view and explained later for FIG. 5b. The camera used was a FLIR Grashopper 3 5MP camera with a TechSpec 0.5x telecentric lens with a width W of 17,6 mm and a height H of 13,2 mm and which gives a total scale of 139 pixels per millimeter. The camera was powered by a 24VDC power supply. For illumination, 2x AL295 lamps from MicroBrite™ Bar Lights with a total power of 80W (800 W overdrive) (LED) were used for enough illumination to have the lowest possible aperture on the lens for maximum depth of field so that the entire volume of the flow cell was in focus. The AL295 lamps used were 89.4 mm long and with white light (5500K to 6100K). The camera was connected to a Raspberry Pi 4 which runs a custom software for setting up the correct camera settings and capturing and storing images from the camera. The Raspberry Pi 4 was powered by a 5VDC power supply. The images were automatically stored on a 128GB memory stick. The speed of the passing larvae was adjusted by a flow valve (not shown). All components were attached to 2 pieces of 10 mm HDPE boards with bracing. Silicone hoses were used to connect a funnel to the flow cell.

FIG. 5b show detailed sketches of the flow cell and mirror with container filled with degassed water from FIG. 5a from the side and front. The flow cell is a square shaped quartz tube with an inner width of 4 mm and is approx. 300 mm long. Two mirrors were arranged at an angle of 22.5° approximately 11 mm below the flow cell to achieve two reflections that are 90° relative to each other. The mirrors and flow cell were located inside a waterproof container filled with degassed water to remove optical distortions due to irregularities in the flow cell. The container was made of Lexan. The container may also be filled with e.g. clear mineral oil. The optics and flow cell were optimized for 3-10 mm long larvae with a maximum diameter of 4 mm.

FIG. 5 shows three images of the same larva taken with the imaging system explained above and shown in FIGS. 5a, 5b. The fish larva in FIG. 5 is a newly hatched zebrafish larva. Approximately 300 newly hatched zebrafish larvae in a water volume of approx. 1 decilitre were flowed through the imaging system. The images on the left and right are images taken via the mirrors on the right and left side and are rotated 90° relative to each other. The image in the middle is taken directly by the zebrafish larva inside the flow cell. As can be seen from the images, the yolk of the zebrafish larvae has different visibility in the images. Also, in one image the zebrafish larvae is quite straight, whereas on another image the tail is curved. By obtaining images from more than one side this increases the chances of being able to measuring e.g. morphometrics of the zebra fish larvae. The yoke may be measured from one image where the yoke is visible, whereas the length may be measured from another image. This enables improved measurement of the size of the yolk as well as other parameters as length of the zebrafish larvae.

The example imaging system is explained for ex-situ imaging of fish larvae. The imaging system may also be an in-vivo system. Also, as explained earlier, the imaging system may also be used for imaging of other small aquatic organisms as e.g. small fish, fish larva algae, crustaceans, zooplankton, or an egg from an aquatic organism. The imaging system may be used for monitoring size or growth of small aquatic organisms. The imaging system may also be used for constructing 3D images of small aquatic organisms, for morphometric measurements of small in 5 aquatic organisms, for monitoring growth of fish larvae, for monitoring a health status of fish larvae, or for biometric measurements of fish larvae or for monitoring of fish larvae in production tanks.

Having described example embodiments of the invention it will be apparent to those skilled in the art that other embodiments incorporating the concepts may be used. These and other non-limiting examples illustrated above are intended by way of example only and the actual scope of the invention is to be determined from the following claims.

1. An imaging system for imaging at least one aquatic organism, the imaging system comprising:
an imaging device, and
at least two mirrors, wherein the mirrors are arranged to provide imaging of the at least one aquatic organism from at least two angles.

2. Imaging system according to claim 1, further comprising at least two illumination sources.

3. Imaging system according to claim 1, wherein the imaging device is arranged on a first side of the at least one aquatic organism and the at least two mirrors are arranged on a second side of the at least one aquatic organism, wherein the first side is opposite the second side.

4. Imaging system according to claim 1, wherein the at least two mirrors are arranged to obtain at least two reflections from the at least one aquatic organism.

5. Imaging system according to claim 1, wherein the at least two mirrors and the imaging device are arranged to obtain a combined image of the at least one aquatic organism from three different angles, where the first and second angles are provided by reflections from the at least one aquatic organism with the at least two mirrors and into the imaging device and the third angle is provided by a direct image of the at least one aquatic organism into the imaging device.

6. Imaging system according to claim 1, wherein the at least two mirrors are each arranged at an angle relative to a plane perpendicular to the axis of a field of view of the imaging device, wherein the angel is approximately from 15° to 45°.

7. Imaging system according to claim 1, wherein the at least two mirrors are arranged approximately 22.5° relative to a plane perpendicular to the axis of a field of view of the imaging device to obtain two reflections from the aquatic organism of approximately 90° relative to each other.

8. Imaging system according to claim 1, wherein each of the at least two mirrors are arranged at an angle of approximately 30° relative to a plane perpendicular to the axis of the field of view of the imaging device.

9. Imaging system according to claim 1, wherein the at least two illumination sources are arranged on opposite sides of the imaging device.

10. Imaging system according to claim 1, wherein the at least two mirrors are arranged to obtain a combined image of the at least one aquatic organism provided by views from three different angles, where the three different views are at an angle of 120° relative to each other, wherein two of the angles are reflections from the aquatic organism via the at least two mirrors and into the imaging device and a third angle is a direct image of the aquatic organism into the imaging device.

11. Imaging system according to claim 1, wherein the at least two mirrors are symmetrically arranged, or nearly symmetrically arranged, relative to the axis of the field of view of the imaging device.

12. Imaging system according to claim 1, wherein the at least two mirrors are arranged to enable a same focus area for the imaging device for the viewing angles.

13. Imaging system according to claim 1, wherein the imaging device comprising a telecentric lens.

14. Imaging system according to claim 1, wherein the at least one aquatic organism is arranged to flow through a flow cell while being imaged by the imaging device.

15. Imaging system according to claim 1, wherein the flow cell and the at least two mirrors are arranged inside a container filled with transparent material, preferably a degassed liquid or a clear resin.

16. Imaging system according to claim 15, wherein the imaging device and the at least two illumination sources are arranged on the inside or the outside of the container.

17. Imaging system according to claim 16, wherein the flow cell is transparent.

18. Imaging system according to claim 15, wherein the container is transparent.

19. Imaging system according to claim 1, wherein the container is made of polycarbonate or quartz.

20. Imaging system according to claim 1, wherein the flow cell is made of polycarbonate, quartz or plastic.

21. Imaging system according to claim 1, wherein the flow cell comprises at least one optical diffuser.

22. Imaging system according to claim 1, wherein the images of the at least two angles/sides of the at least one aquatic organism are obtained simultaneously.

23. Flow-through imaging system for imaging at least one aquatic organism while the at least one aquatic organism passes through a flow cell, the system comprising:
a flow cell, and
an imaging system according to claim 1.

24. Flow-through imaging system according to claim 23, wherein the flow cell and the at least two mirrors are arranged inside a container filled with a liquid or clear resin to remove optical distortions caused by irregularities in the flow cell.

25. Flow-through imaging system according to claim 23, further comprising a valve for adjustment of a flow through the flow cell.

26. Use of the imaging system according to claim 1 for monitoring size or growth of small aquatic organisms.

27. Use of the imaging system according to claim 1 for constructing 3D images of small aquatic organisms.

28. Use of the imaging system according to claim 1 for morphometric measurements of small aquatic organisms.

29. Use of the flow-through imaging system according to one claim 23 for monitoring growth of fish larvae, or for monitoring a health status of fish larvae, or for biometric measurements of fish larvae or for monitoring of fish larvae in production tanks.

30. Use according to claim 26, wherein the small aquatic organism is at least one of a fish larva, zebrafish larva, algae, crustacean, zooplankton or an egg from an aquatic organism.

31. Use of the flow-through imaging system according to claim 23 for constructing 3D images of small aquatic organisms.

32. Use of the flow-through imaging system according to claim 23 for morphometric measurements of small aquatic organisms.
